(19) United States
(12) Patent Application Publication
(10) Pub. No.: US 2011/0130434 A1
(43) Pub. Date: Jun. 2, 2011

(54) MODIFIED RELEASE RAMIPRIL COMPOSITIONS AND USES THEREOF

(75) Inventors: Rajesh Jain, New Delhi (IN); Sarabjit Singh, Navi Mumbai (IN)

(73) Assignee: Panacea Biotec Ltd., Navi Mumbai, Maharashtra (IN)

(21) Appl. No.: 13/055,778

(22) PCT Filed: Jul. 28, 2009
(86) PCT No.: PCT/IN2009/000428

§ 371 Date: Jan. 25, 2011

(30) Foreign Application Priority Data
Jul. 30, 2008 (IN) 1621/MUM/2008

(51) Int. Cl. A61K 31/403; A61P 9/12

(52) U.S. Cl. 514/412

(57) ABSTRACT

The invention relates to a modified release pharmaceutical composition comprising ramipril or pharmaceutically acceptable salts thereof and pharmaceutically acceptable excipient(s) wherein pharmaceutical composition is bioequivalent to conventional immediate release formulation of ramipril administered twice daily. The invention further relates to a modified release pharmaceutical composition comprising: an immediate release component comprising ramipril or pharmaceutically acceptable salts thereof and a modified release component comprising ramipril or pharmaceutically acceptable salts thereof. The invention further relates to the process for the preparation of modified release pharmaceutical composition of ramipril.

### FIELD OF THE INVENTION

The present invention relates to a modified release pharmaceutical composition comprising ramipril or pharmaceutically acceptable salts thereof and pharmaceutically acceptable excipient. The present invention further relates to a modified release pharmaceutical composition comprising ramipril or pharmaceutically acceptable salts thereof and a pharmaceutically acceptable excipient wherein pharmaceutical composition is bioequivalent to conventional immediate release formulation of ramipril (ALTACE®) administered twice daily.

### BACKGROUND OF THE INVENTION

Ramipril, (2S,3aS,6aS)-1[(S)—N—[(S)-1-carboxy-3-phenylpropyl]alanyl]octahydrocycl-openta[b]lpyrrole-2-carboxylic acid, 1-ethyl ester is an angiotensin converting enzyme (ACE) inhibitor.

Ramipril (disclosed in EP 079022) is a long-acting ACE inhibitor. Its active metabolite is the free acid ramiprilat, which is obtained in vivo upon administration of ramipril. In hypertensive patients administration of ramipril is known to cause a reduction in peripheral arterial resistance and thus a reduction of the blood pressure without a compensatory rise in heart rate. It is being used in the treatment of hypertension and congestive heart failure. Furthermore, ramipril has been shown to reduce mortality in patients with clinical signs of congestive heart failure after surviving an acute myocardial infarction. Ramipril has been suggested to have an added advantage over many other ACE inhibitors due to its pronounced inhibition of ACE in tissues resulting in organ protective effects in e.g. the heart, lung, and kidney.

The recommended initial dose of ramipril for patients having hypertension, not receiving a diuretic, is 2.5 mg once a day. Dosage should be adjusted according to the blood pressure response. The usual maintenance dosage range is 2.5 to 20 mg per day administered as a single dose or in two equally divided doses (twice daily or twice a day). It is critical to note that in some patients treated once daily, the antihypertensive effect may diminish toward the end of the dosing interval. In such patients, an increase in dosage or twice daily administration should be considered.

It is known, in Chronobiologia, Vol. 13, pages 239 to 243, (1986), that blood pressure has within-day rhythmicity, and that the highest pressure values are seen often in the morning hours just after waking by the patient. The rise in blood pressure occurring at waking requires a dosage form that is administered on retiring and delivers its drug after a drug-free interval during sleep. This drug delivery pattern provides the need for therapy at the appropriate time, thereby substantially lessening the incidence of a waking elevated blood pressure.

U.S. Pat. No. 5,785,994 relates to a dosage form provided as an osmotic device comprising means for the rate-programmed delivery of a drug in time-varying patterns to a drug recipient. The patent describes an osmotic drug delivery device comprising means for delivering a pulsed dose of drug to a human, means for providing a drug-free interval, and then providing a recurring pulse dose of drug to the human. The patent discloses osmotic drug delivery device for delivering calcium channel blockers for lessening the incidence of waking elevated blood pressure, when administered at the time of retiring.

U.S. Pat. No. 6,764,697 relates to a dosage form that after administration of the dosage form is followed by a drug-free period, which dosage form at this later time delivers a dose of drug for delayed therapy. The patent also relates to a method of delayed-drug therapy by administering a dosage form comprising a drug composition and a second composition (osmotic composition) that delays the onset of drug delivery and after the drug-free interval delivers a drug for its therapeutic effect to produce the effect at morning hours, when administered before retiring. The patent discloses the use of water-soluble non-ionic polymer for providing delayed therapy of verapamil.

Osmotic drug delivery system is a very complicated and costly technology for providing the controlled release of the active agent. The technology involves highly sophisticated instrumentation and especially skilled personnel resulting into increased cost.

U.S. Pat. No. 6,500,459 describes a pharmaceutical compositions that delays the release of drug from a pharmaceutical compositions for a predetermined time or provide a controlled onset after administration of the pharmaceutical compositions and continue releasing the same drug from the pharmaceutical compositions at a predetermined release rate such that the blood levels achieved by such pharmaceutical compositions provide a significant therapeutic benefit to patients suffering from various disease states. The patent discloses the pharmaceutical composition for providing controlled onset and sustained release of verapamil using a functional coating membrane for controlled onset and the hydrophilic polymeric matrix for the sustained release.

U.S. Pat. No. 6,267,990 relates to the pharmaceutical preparation comprising initial dose of ACE inhibitor in a composition; first delayed release pellets comprising ACE inhibitor and excipients, covered with a coating and a second delayed release pellet comprising ACE inhibitor and excipients, covered with a coating, wherein the amount of coatings on first and second delayed release pellets are present in a ratio, based on weight, within the range of from 1:2 to 1:7. The pharmaceutical preparation permits the controlled release of ACE inhibitor more particularly captopril and thus ensures a therapeutically effective blood level over a prolonged period with minimal variations in the blood level concentration. Pelletization is a very costly and time consuming technology and requires very sophisticated instrumentation.

The prior arts described above have used the complex and costly technologies to address the problem of waking hour elevated blood pressure; to produce the effect in morning hours and to minimise the variation, with calcium channel blockers such as verapamil and also captopril, but none of the prior arts seems to address the most critical requirement of providing extended therapeutic plasma level of ramipril or its active metabolite i.e. the higher plasma concentration of ramipril or its active metabolite between 20-24 hours after dosing, such that the antihypertensive effect does not diminish towards the end of the dosing interval or the composition is capable of effectively reducing the blood pressure between dosing interval.

Thus, there is a continuing need in the art for a relatively simple and economical modified release pharmaceutical composition of ramipril, which is further suitable for effective once a day administration.

The inventors of the present invention have found that the higher levels of ramipril or its active metabolite can be achieved by modifying the release of ramipril from the composition and providing a release profile such that the plasma concentration of ramipril or its active metabolite is higher towards the end of the dosing interval when compared with the marketed formulation of ramipril (ALTACE®) administered once daily and preparing a composition which is bioequivalent to the conventional immediate release formulation of ramipril administered twice daily, using a much simpler and cost effective technology. The extended therapeutic plasma level of ramipril or its active metabolite or higher plasma concentration of ramipril or its active metabolites between 20-24 hours after dosing provide effective once daily dosage regimen and increased patient compliance.

### SUMMARY OF THE INVENTION

The present invention relates to a modified release pharmaceutical composition comprising ramipril or pharmaceutically acceptable salts thereof and pharmaceutically acceptable excipient(s).

The present invention further relates to a modified release pharmaceutical composition comprising ramipril or pharmaceutically acceptable salts thereof and pharmaceutically acceptable excipient(s) wherein pharmaceutical composition is bioequivalent to conventional immediate release formulation of ramipril administered twice daily.

The present invention further relates to a modified release pharmaceutical composition comprising ramipril or pharmaceutically acceptable salts thereof and pharmaceutically acceptable excipient(s) wherein pharmaceutical composition is bioequivalent to conventional immediate release formulation of ramipril administered twice daily and provides extended therapeutic plasma level over 24 hours.

The present invention relates to a modified release pharmaceutical composition comprising ramipril or pharmaceutically acceptable salts thereof and pharmaceutically acceptable excipient(s), wherein the composition exhibits following dissolution profile: not more than 80% of drug is released in 2 hours and atleast about 15% of drug is released within 6hours, when tested by a pH Change dissolution method in USP Type II apparatus at 50 rpm.

The present invention further relates to the process for the preparation of modified release pharmaceutical composition comprising ramipril or pharmaceutically acceptable salts thereof and pharmaceutically acceptable excipient(s).

The present invention further relates to a modified release pharmaceutical composition comprising ramipril or pharmaceutically acceptable salts thereof and pharmaceutically acceptable excipient(s), wherein the composition provides average plasma concentration of ramiprilat of atleast about 4.0 ng/ml at 24 hours after single dose administration of 10mg ramipril.

A modified release pharmaceutical composition comprising ramipril or pharmaceutically acceptable salts thereof and a release modifying material and optionally one or more pharmaceutically acceptable excipient(s), wherein the composition provides average plasma concentration of ramiprilat at 24 hours comparable with that of the marketed ramipril formulation administered twice daily.

### BRIEF DESCRIPTION OF DRAWINGS:

FIG. 1 shows the In-Vitro Dissolution profile for Example-1 in pH Change Dissolution Method Using USP Type II Apparatus at 50 rpm.

FIG. 2 shows the In-Vitro Dissolution profile for Example-5 in pH Change Dissolution Method Using USP Type II Apparatus at 50 rpm.

FIG. 3 shows the Mean Plasma Concentration of Ramipril Vs Time Plot for Test and Reference product in a Fed Study

FIG. 4 shows the Mean Plasma Concentration of Ramiprilat Vs Time Plot for Test and Reference product in a Fed Study

FIG. 5 shows the Mean Plasma Concentration of Ramipril Vs Time Plot for Test and Reference product in a Fasting Study

FIG. 6 shows the Mean Plasma Concentration of Ramiprilat Vs Time Plot for Test and Reference product in a Fasting Study

FIG. 7 shows the Mean Plasma Concentration of Ramipril Vs Time Plot for Test and Reference product in a Steady State Study.

FIG. 8 shows the Mean Plasma Concentration of Ramiprilat Vs Time Plot for Test and Reference product in a Steady State Study.

### DETAILED DESCRIPTION OF THE INVENTION:

Ramipril is a long-acting ACE inhibitor. Its active metabolite is the free acid ramiprilat, which is formed in vivo upon administration of ramipril. In hypertensive patients administration of ramipril is known to cause a reduction in peripheral arterial resistance and thus a reduction of the blood pressure without a compensatory rise in heart rate.

Ramipril has a dosage regimen of administering once daily, but it has been observed that the antihypertensive effect of ramipril diminishes towards the end of dosing interval in once daily dosage regimen. In case when the antihypertensive effect of ramipril diminishes towards the end of dosing interval i.e. between 20-24 hours after dosing, then doctor prescribes the patient with increased dose of ramipril or administration in two equally divided doses (twice daily administration or twice a day administration).

Ramipril after oral administration gets metabolized to its active diacid metabolite ramiprilat having 6 times the ACE inhibitory activity of ramipril and has a half life of 13-17 hours. The longer half life of ramiprilat is responsible for the once daily administration of ramipril to provide the antihypertensive effect. The lower level of plasma concentration of ramipril or its active metabolites achieved at 20-24 hour after dosing can be sole reason for diminishing of antihypertensive effect towards dosing interval and resulting in the prescription of twice daily administration of dosage form.

The pharmaceutical composition which is bioequivalent to the conventional immediate release formulation of ramipril administered twice daily and provides the extended therapeutic plasma level i.e. higher plasma concentration of ramiprilat at 20 to 24 hours after dosing, to that obtained from the once daily administration of marketed ramipril formulation (ALTACE®) will lead to effective once a day administration of ramipril.

The higher plasma concentration of ramiprilat can be achieved by providing a modified release composition which releases ramipril in such a manner that the conversion of ramipril to ramiprilat is delayed or prolonged and thus results in the higher efficacy.

The modified release formulation which is bioequivalent to the conventional immediate release formulation of ramipril administered twice daily will provide the effective plasma concentration as achieved by the twice daily administration and thus increases the patient compliance and minimise the variations.

The present invention further relates to a modified release pharmaceutical composition comprising ramipril or pharmaceutically acceptable salts thereof and pharmaceutically acceptable excipient(s) wherein pharmaceutical composition is bioequivalent to conventional immediate release formulation of ramipril administered twice daily.

The present invention further relates to a modified release pharmaceutical composition comprising ramipril or pharmaceutically acceptable salts thereof and pharmaceutically acceptable excipient(s) wherein pharmaceutical composition is bioequivalent to conventional immediate release formulation of ramipril administered twice daily and provides extended therapeutic plasma level of ramiprilat over 24 hours.

Conventional immediate release formulation of ramipril can be any immediate release formulation of ramipril commercially available in market such as ALTACE® (King Pharmaceuticals)

Two compositions can be considered as “bioequivalent” if the 90% Confidence Interval of the relative mean Cₘₐₓ; and AUC of the test to reference is within 80.00% to 125.00%.

The present invention relates to a modified release pharmaceutical composition comprising ramipril or pharmaceutically acceptable salts thereof and pharmaceutically acceptable excipient(s), wherein the composition exhibits following dissolution profile: not more than 80% of drug is released in 2 hours and atleast about 15% of drug is released within 6 hours, when tested by a pH Change dissolution method in USP Type II apparatus at 50 rpm.

The modified release pharmaceutical composition of the present invention provides the release profile such that the concentration of ramipril or its active metabolite is maintained for a prolonged period of time. The prolonged presence of ramipril or its active metabolites in the plasma results in the effective once a day administration of ramipril dosage form as compared to the marketed ramipril formulation (ALTACE®).

The present invention further relates to a modified release pharmaceutical composition comprising ramipril or pharmaceutically acceptable salts thereof and pharmaceutically acceptable excipient(s), wherein the composition exhibits following dissolution profile: not more than 75% of drug is released in 2 hours and atleast about 20% of drug is released within 6 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: not more than 70% of drug is released in 2 hours and atleast about 25% of drug is released within 6 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: not more than 65% of drug is released in 2 hours and atleast about 30% of drug is released within 6 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: not more than 60% of drug is released in 2 hours and atleast about 35% of drug is released within 6 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: not more than 60% of drug is released in 3 hours and atleast about 40% of drug is released within 6 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: not more than 60% of drug is released in 3 hours and atleast about 40% of drug is released within 4 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: not more than 60% of drug is released in 3 hours and atleast about 40% of drug is released within 3 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: atleast about 40% of drug is released within 2 hours and not more than 60% of drug is released in 3 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: atleast about 40% of drug is released within 1 hours and not more than 60% of drug is released in 3 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: atleast about 40% of drug is released within 1 hours, not more than 60% of drug is released in 5 hours and atleast about 80% is released at 9 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: atleast about 35% of drug is released within 30 min, not more than 60% of drug is released in 5 hours and atleast about 80% is released at 9 hours, when tested by a pH Change dissolution method in USP Type II apparatus at 50 rpm.

The present invention further relates to a modified release pharmaceutical composition comprising ramipril or pharmaceutically acceptable salts thereof and pharmaceutically acceptable excipient(s), wherein the composition exhibits following dissolution profile: atleast about 30% of drug is released within 30 min, not more than 55% of drug is released in 5 hours and atleast about 80% is released at 9 hours, when tested by a pH Change dissolution method in USP Type II apparatus at 50 rpm.

‘pH Change dissolution method’ as used in this context means carrying out the dissolution of the dosage form in pH 1.2 for 2 hrs, followed by pH 4.5 for 1 hr, pH 6.8 for 3 hrs and further dissolution in pH 7.4.

The present invention further relates to a modified release pharmaceutical composition comprising ramipril or pharmaceutically acceptable salts thereof and pharmaceutically acceptable excipient(s), wherein the composition provides average plasma concentration of ramiprilat of atleast about 4.0 ng/ml at 24 hours after single dose administration.

The present invention further relates to a modified release pharmaceutical composition comprising ramipril or pharmaceutically acceptable salts thereof and pharmaceutically acceptable excipient(s), wherein the composition provides average plasma concentration of ramiprilat of atleast about 4.0 ng/ml at 24 hours after single dose administration of 10mg ramipril.

The present invention further relates to a modified release pharmaceutical composition comprising ramipril or pharmaceutically acceptable salts thereof and pharmaceutically acceptable excipient(s), wherein the composition provides average plasma concentration of ramiprilat of atleast about 4.7 ng/ml at 20 hours after single dose administration of 10 mg ramipril.

The present invention further relates to a modified release pharmaceutical composition comprising ramipril or pharmaceutically acceptable salts thereof and pharmaceutically acceptable excipient(s), wherein the composition provides peak plasma concentration of ramiprilat of atleast from about 4.0 ng/m1 to about 40 ng/ml after administration of 10 mg ramipril.

The present invention further relates to a modified release pharmaceutical composition comprising ramipril or pharmaceutically acceptable salts thereof and pharmaceutically acceptable excipient(s), wherein the composition provides peak plasma concentration of ramiprilat of atleast from about 8.0 ng/ml to about 30 ng/ml after administration of 10 mg ramipril.

The present invention further relates to a modified release pharmaceutical composition comprising ramipril or pharmaceutically acceptable salts thereof and pharmaceutically acceptable excipient(s), wherein peak plasma concentration of ramiprilat is achieved between 1 to 12 hours after single dose administration of 10 mg ramipril.

The average plasma concentration of ramiprilat at 24 hours achieved after single dose administration of the modified release pharmaceutical composition of the present invention comprising 10 mg ramipril is atleast about 4.0 ng/ml. Preferably, the average plasma concentration can be in the range from about 4.0 ng/m1 to about 40 ng/ml.

The average plasma concentration of ramiprilat at 20 hours achieved after single dose administration of the modified release pharmaceutical composition of the present invention comprising 10 mg ramipril is atleast about 4.7 ng/ml. Preferably, the average plasma concentration can be in the range from about 4.7 ng/ml to about 40 ng/ml.

The peak plasma concentration of ramiprilat (Cmax) achieved after administration of the modified release pharmaceutical composition of the present invention can be in the range from about 4.0ng/m1 to about 40ng/ml, preferably in the range from about 5.0 ng/ml to about 30 ng/ml.

The peak plasma concentration of ramiprilat achieved after single dose administration of the modified release pharmaceutical composition of the present invention is achieved between 1 to 12 hours after single dose administration of 10 mg ramipril.

The present invention further relates to a modified release pharmaceutical composition comprising ramipril or pharmaceutically acceptable salts thereof and a release modifying material and optionally one or more pharmaceutically acceptable excipient(s), wherein the composition provides average plasma concentration of ramiprilat at 24 hours comparable with that of the marketed ramipril formulation administered twice daily.

The term “comparable” means average plasma concentration of ramiprilat wherein the ratio of average plasma concentration of ramiprilat for the test (formulation as per the present invention) to reference (ALTACE®) is within or equal to 0.8 to 1.25.

In a preferred embodiment a modified release pharmaceutical composition comprising ramipril or pharmaceutically acceptable salts thereof and a release modifying material and optionally one or more pharmaceutically acceptable excipient(s), wherein the composition provides average plasma concentration of ramiprilat at 24 hours comparable with that of the marketed ramipril formulation administered twice a day in divided doses and provides the average plasma concentration of ramiprilat of atleast about 4.0 ng/ml at 24 hours after administration of 10 mg ramipril at steady state.

In another aspect, the present invention relates to a modified release pharmaceutical composition comprising ramipril or pharmaceutically acceptable salts thereof and a release modifying material optionally in combination with one or more pharmaceutically acceptable excipient, wherein the composition is optionally coated with a coating.

In another aspect, the present invention relates to a modified release pharmaceutical composition comprising ramipril or pharmaceutically acceptable salts thereof in an amount from about 1% to about 40% w/w of the composition and one or more pharmaceutically acceptable excipient in an amount from about 1% to about 99% w/w of the composition selected from the group comprising diluents, carriers, fillers, release modifying material, bulking agents, binders, disintegrants, polymer, lubricant, glidant, surface active agents, stabilizers, absorption accelerators, flavoring agents, preservatives, antioxidants, buffering agents and combinations thereof.

In another aspect, the present invention relates to a modified release pharmaceutical composition comprising ramipril or pharmaceutically acceptable salts thereof in an amount from about 1% to about 30% w/w of the composition and one or more pharmaceutically acceptable excipient in an amount from about 5% to about 95% w/w of the composition selected from the group comprising diluents, carriers, fillers, release modifying material, bulking agents, binders, disintegrants, polymer, lubricant, glidant, surface active agents, stabilizers, absorption accelerators, flavoring agents, preservatives, antioxidants, buffering agents and combinations thereof.

As used herein the term ‘modified release’ means but not limited to sustained release, controlled release, retarded release, timed release, programmed release, burst release, pulsed release, prolonged release, delayed release, immediate release, slow release, extended release or combinations thereof. Preferably modified release is sustained release or pulsed release, more preferably is pulsed release.

As used herein the term “sustained release” composition means, but not limited to, the composition which provides the slow release of drug present in the composition. Preferably the component provides the slow release of the drug upto 16 hours, more preferably upto 12 hours.

As used herein, term “pulsed release” composition means any pulsed release composition that releases the drug in two or more pulses.

Another aspect of the present invention relates to a modified release pharmaceutical composition comprising:
a) an immediate release component comprising ramipril or pharmaceutically acceptable salts thereof and one or more pharmaceutically acceptable excipient;
b) a modified release component comprising ramipril or pharmaceutically acceptable salts thereof and one or more pharmaceutically acceptable excipient;
c) optionally a sustained release component comprising ramipril or pharmaceutically acceptable salt thereof and one or more pharmaceutically acceptable excipient.

As used here in the term “modified release component” means but not limited to, the composition which provides the modified release of the drug incorporated in the component.

Another aspect of the present invention relates to a modified release pharmaceutical composition comprising:
a) an immediate release component comprising ramipril or pharmaceutically acceptable salts thereof and one or more pharmaceutically acceptable excipient;
b) a modified release component comprising ramipril or pharmaceutically acceptable salts thereof and one or more pharmaceutically acceptable excipient;
c) optionally a sustained release component comprising ramipril or pharmaceutically acceptable salt thereof and one or more pharmaceutically acceptable excipient.
   wherein Tmax from an immediate release component i.e. Tₘₐₓ₁ ranges from about 1 hour to about 4 hours and Tmax from a modified release component i.e. Tₘₐₓ₂ ranges from about 6 hours to about 10 hours.

‘Tₘₐₓ’ as used herein means the time to achieve maximum plasma concentration after administration of the modified release pharmaceutical composition. ‘Tₘₐₓ₁’ and ‘Tₘₐₓ₂’ when used indicates the time to achieve maximum concentration of ramiprilat from immediate release component and time to achieve maximum concentration of ramiprilat from modified release component respectively on administration of the modified release pharmaceutical composition of ramipril in a fasting condition, wherein the two peaks are distinct. Tₘₐₓ₁ can range from about 1 hour to about 4 hours, preferably from about 1.5 hours to about 3.5 hours and Tₘₐₓ₂ can range from about 6 hours to about 10 hours, preferably from about 7 hours to about 9.5 hours.

Another aspect of the present invention relates to a modified release pharmaceutical composition comprising:
a) an immediate release component comprising ramipril or pharmaceutically acceptable salts thereof and one or more pharmaceutically acceptable excipient;
b) a delayed release component comprising ramipril or pharmaceutically acceptable salts thereof and one or more pharmaceutically acceptable excipient;
c) optionally a sustained release component comprising ramipril or pharmaceutically acceptable salt thereof and one or more pharmaceutically acceptable excipient.

In another aspect the present invention relates to a modified release pharmaceutical composition comprising:
a) an immediate release component comprising ramipril or pharmaceutically acceptable salts thereof and one or more pharmaceutically acceptable excipient;
b) a sustained release component comprising ramipril or pharmaceutically acceptable salts thereof and a release modifying polymeric carrier optionally in combination with one or more pharmaceutically acceptable excipient.

The term “Immediate release component” according to the present invention is the composition which provides the release of substantially complete amount of drug present in the immediate release component within 2 hours. The immediate release component comprises ramipril or pharmaceutically acceptable salt thereof in an amount from about 30% to about 80% of the total amount of ramipril present in the modified release pharmaceutical composition, preferably in an amount from about 40% to 60% of the total amount of ramipril present in the modified release pharmaceutical composition.

The term “delayed release component” according to the present invention is the composition which delays the release of drug present in the component for atleast about 3 hours. The delayed release of the drug can be achieved by using various release modifying polymers in the matrix or in the coating. The delayed release component comprises ramipril or pharmaceutically acceptable salt thereof in an amount from about 20% to about 70% of the total amount of ramipril present in the modified release pharmaceutical composition, preferably in an amount from about 30% to about 60% of the total amount of ramipril present in the modified release pharmaceutical composition.

The term sustained release component” according to the present invention is the composition which provides the slow release of drug present in the component. Preferably the component provides the slow release of the drug upto 16 hours, more preferably upto 12 hours. The sustained release component comprises ramipril or pharmaceutically acceptable salt thereof in an amount from about 0% to about 30% of the total amount of ramipril present in the modified release pharmaceutical composition, preferably in an amount from about 10% to about 20% of the total amount of ramipril present in the modified release pharmaceutical composition.

The pharmaceutical composition according to the present invention can be prepared by any suitable process known in the art. Preferably the pharmaceutical composition of the present invention can be prepared by direct compression, dry granulation or wet granulation.

The present invention further relates to a process for the preparation of modified release pharmaceutical composition comprising ramipril or pharmaceutically acceptable salts thereof, comprising the steps of:
a) blending ramipril or its pharmaceutically acceptable salts thereof with one or more pharmaceutically acceptable excipient.
b) Compressing the blend of step a) to form the tablet or filling the blend of step a in a capsule.
c) Optionally dividing the total number of tablets of step b) into two equal parts and coating the one part of tablets with a delayed release coating.
d) Optionally filling one coated and one uncoated tablet in a capsule.

The present invention further relates to a process for the preparation of modified release pharmaceutical composition comprising ramipril or pharmaceutically acceptable salts thereof, comprising the steps of:
a) blending ramipril or its pharmaceutically acceptable salts thereof with one or more pharmaceutically acceptable excipient.
b) Granulating the blend of step a)
c) Compressing the granules of step b) to form tablet or filling the granules in a capsule.
d) Optionally dividing the total number of tablets of step c) into two equal parts and coating the one part of tablets with a delayed release coating.
e) Optionally filling one coated and one uncoated tablet in a capsule.

The present invention further relates to the method of treating cardiovascular events in mammals comprising administering a modified release pharmaceutical composition comprising ramipril or pharmaceutically acceptable salts thereof.

As used herein the term ‘cardiovascular events’ means, but not limited to, hypertension, myocardial infarction, diabetes, left ventricular dysfunction, heart failure, cardiac insufficiency, stroke, congestive heart failure, worsening of angina, cardiovascular death, overt nephropathy in diabetic patients, peripheral vascular disease or the like.

In another aspect, the present invention relates to a modified release pharmaceutical composition comprising ramipril or pharmaceutically acceptable salts thereof and pharmaceutically acceptable excipient(s), wherein the composition effectively reduces the blood pressure between dosing intervals.

In yet another aspect, the present invention relates to method of reducing cardiovascular morbidity and mortality in mammals comprising administering a modified release pharmaceutical composition comprising ramipril or pharmaceutically acceptable salts thereof and pharmaceutically acceptable excipient(s), wherein the composition effectively reduces the blood pressure between dosing intervals.

It must be noted that as used in the specification and the appended claims, the singular forms ‘a’, ‘an’ and ‘the’ include plural references unless the context clearly indicates otherwise. Thus for example, use of the term ‘an active agent’ includes reference to one or more active agents.

The term ‘pharmaceutical composition’ according to present invention can be a mixture of active ingredient optionally in combination with pharmaceutically acceptable excipient processed by any method known in the art.

The pharmaceutical composition can be formulated in any dosage form which can deliver or release the drug in-vitro as well as in-vivo through any route of administration like oral, buccal, sublingual, nasal, transmucosal or the like, preferably by oral route like tablets, capsules, mini-tablets, oral films, sprays, pellets, granules or powder or the like or combinations thereof. The combination of dosage forms such as tablet in tablet, inlay tablet, minitablet enclosed in capsule, pellets enclosed in capsule, granules enclosed in capsule, powder enclosed in capsule, bilayer tablet, multilayer tablet, capsule coated with active ingredient or the like. More preferably the pharmaceutical composition of the present invention can be selected from the group comprising pellets enclosed in capsule, minitablet enclosed in capsule, tablet enclosed in capsule, bilayer or multilayer tablets, and granule enclosed in capsule.

As disclosed herein and as used in the compositions and methods of the present invention, the term ‘ramipril’, includes ramipril, its pharmaceutically acceptable salts, conjugates, polymorphs, derivatives, complexes, prodrugs and natural and synthetic analogues, solvate, hydrate or anhydrates and combinations thereof. Use of the term ‘drug’ or ‘active ingredient’ in context of the present invention refers to ramipril including the forms mentioned herewith.

The modified release pharmaceutical composition may comprise about 2.5 to 20 mg of the ramipril or pharmaceutically acceptable salts thereof. Preferably, the modified release pharmaceutical composition comprises about 1.25 mg, 2.5 mg, 5 mg, 10 mg or 20 mg of the derivative. Preferably, the active ingredient can be present in an amount from about 1% to about 40% w/w of the modified release pharmaceutical composition, more preferably in an amount from about 1% to about 30% w/w of the modified release pharmaceutical composition.

The term ‘about’ with respect to the composition can mean plus or minus a range of up to 20%, preferably up to 10%, more preferably up to 5%. This term particularly with respect to biological systems or processes can mean within an order of magnitude, preferably within 5-fold, and more preferably within 2-fold, of a value. Where particular values are described in the application and claims, unless otherwise stated the term “about” means within an acceptable error range for the particular value as determined by one ordinary skilled in the art.

The term ‘pharmaceutically acceptable excipient’ according to the present invention means, but not limited to, any one or more inactive ingredient which is required for the composition of ramipril in a suitable dosage form. Particularly the excipient includes, but not limited to, diluents, carriers, fillers, bulking agents, binders, disintegrants, polymer, lubricant, glidant, surface active agents, stabilizers, absorption accelerators, flavoring agents, preservatives, antioxidants, buffering agents, release modifying material, coating material and any other excipient commonly used in the pharmaceutical industry. Said excipients are preferably contained in a percentage from about 1% to about 99%, preferably from about 5% to about 96% or from about 10 to about 96% or from about 15% to about 95% w/w of the modified release pharmaceutical composition.

Diluents increase the bulk of a solid pharmaceutical composition, and may make a pharmaceutical dosage form containing the composition easier for the patient and care giver to handle. Diluents used in the composition include diluents commonly used in solid pharmaceutical compositions. Diluents include, but are not limited to, calcium carbonate, calcium phosphate (dibasic or tribasic), calcium sulfate anhydrous, calcium sulfate hydrate, dextrates, dextrin, dextrose excipient, fructose, kaolin, lactitol, anhydrous lactose, lactose monohydrate, maltose, mannitol, sorbitol, sucrose, starch, pregelatinized starch, talc and the like or combinations thereof. Said diluents are preferably contained in a percentage from about 0% to about 99%, more preferably from 40% to about 96% or from about 50 to about 96% or from about 60% to about 95% w/w of the modified release pharmaceutical composition. Preferably the diluent is calcium sulfate dihydrate or pregelatinized starch or combinations thereof in an amount from about 40 to about 96%.

Carriers for use according to the present invention may include, but are not limited to, hydrophilic or hydrophobic polymeric carriers, lactose, white sugar, sodium chloride, glucose, urea, lipophilic materials, starch, calcium carbonate, calcium sulphate, kaolin, crystalline cellulose, silicic acid, and the like or combinations thereof. Said carriers are preferably contained in a percentage from about 0% to about 99%, more preferably from about 40% to about 96% or from about 50 to about 96% or from about 60% to about 95% w/w of the modified release pharmaceutical composition. Preferably the carrier is calcium sulfate or lactose or combinations thereof in an amount from about 40 to about 96%.

Binders help to bind the active ingredient and other excipients together. Binders used in the composition include binders commonly used in solid pharmaceutical compositions. Binders include, but are not limited to, acacia, alginic acid, carbomer, sodium carboxymethyl cellulose, dextrin, ethylcellulose, gelatin, glucose, guar gum, hydroxypropyl methyl cellulose, hydroxypropyl cellulose, maltose, methylcellulose, povidone, starch, gelatin, methylcellulose, polyethylene oxide and the like or combinations thereof. Said binders are preferably contained in a percentage from about 0% to about 60%, more preferably from about 10% to about 50% or from about 20 to about 40% or from about 25% to about 35% w/w of the modified release pharmaceutical composition. Preferably the binder is hydroxypropyl methyl cellulose or povidone or combinations thereof in an amount from about 10% to about 50%.

Disintegrants can increase dissolution. Examples of suitable disintegrants are starch, pregelatinized starch, sodium starch glycolate, sodium carboxymethyl cellulose, crosslinked sodium carboxymethyl cellulose, clays, microcrystalline cellulose, alginates, gums, surfactants, effervescent mixtures, hydrous aluminum silicate, cross-linked polyvinyl pyrrolidone, and others as known in the art or combinations thereof. Said disintegrants are preferably contained in a percentage from about 10% to about 55%, more preferably from about 15% to about 55% or from about 20 to about 50% w/w of the modified release pharmaceutical composition. Preferably the disintegrant is pregelatinized starch in an amount from about 15 to about 55%.

Stabilizer increases the stability of ramipril w.r.t degradation into inactive metabolites. Stabilizers used in the composition include stabilizers commonly used in solid pharmaceutical compositions. Examples of suitable stabilizers include, but not limited to, buffering agents including tromethamine, alkalizing agents, amino acid or its salts, cellulose or its derivatives, colloidal silicon dioxide, silica, ascorbic acid, organic acids, glutamic acid, betaine hydrochloride, tartaric acid and the like or combinations thereof. Said stabilizers are preferably used in a percentage from about 0% to about 25%, more preferably from about 0.2% to about 20% or from about 0.5% to about 15% w/w of the modified release pharmaceutical composition. Preferably the stabilizer is a buffering agent in an amount from about 0.2% to about 20%.

A lubricant can be added to the composition for ease in processing, e.g., to reduce adhesion to the equipment used during processing, and to ease release of the product from a punch or dye during tableting. Lubricants used in the composition include those commonly used in solid pharmaceutical compositions, including, e.g., calcium stearate, glyceryl behenate, magnesium stearate, mineral oil, polyethylene glycol, sodium stearyl fumarate, stearic acid, talc, vegetable oil, sodium lauryl sulfate, and zinc stearate or the combinations thereof. Said lubricants are preferably contained in a percentage from about 0% to about 5%, more preferably from about 0.2% to about 3% or from about 0.5% to about 3% w/w of the modified release pharmaceutical composition. Preferably the lubricant is magnesium stearate in an amount from about 0.2 to about 3%.

Glidants can be added to improve the flowability of a pharmaceutical composition and improve the accuracy of dosing. Glidants used in the composition include glidants commonly used in solid pharmaceutical compositions, including, e.g., colloidal silicon dioxide, magnesium trisilicate, powdered cellulose, starch, talc, and tribasic calcium phosphate and the like or combinations thereof. Said glidants are preferably contained in a percentage from about 0% to about 5%, more preferably from about 0.2% to about 3% or from about 0.5% to about 3% w/w of the modified release pharmaceutical composition. Preferably the glidant is talc or colloidal silicon dioxide or combinations thereof in an amount from about 0.2 to about 3%.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid and the like or combinations thereof.

Modified release of the drug from the pharmaceutical composition can be achieved by using various release modifying materials of different viscosity and physical properties or by coating the pharmaceutical composition with release modifying material. The pharmaceutical composition where modified release is desired one may employ release modifying material in a matrix system or coating the composition with release modifying material or any other method known in the art. Preferably release modifying materials can be release modifying matrix material or a release modifying coating material.

Release modifying matrix material of the pharmaceutical composition can be one or more selected from the group comprising natural polymers, synthetic polymers, semi-synthetic polymers, hydrophilic polymers or hydrophobic polymers or waxes or combinations thereof, preferably hydrophilic polymers or hydrophobic polymers or combinations thereof. Said release modifying polymeric material are preferably contained in a percentage from about 0% to about 50%, more preferably from about 0.5% to about 45% or from about 2% to about 45% or from about 3% to about 40% w/w of the modified release pharmaceutical composition.

The hydrophilic polymers constituting the modified release polymeric carrier preferably release the active ingredient(s) gradually, slowly or continuously. They swell upon contact with aqueous fluid following administration, resulting in a viscous, drug release regulating gel layer. Hydrophilic polymers suitable for use in this invention are either water soluble or water swellable, and include one or more natural or partially or totally synthetic anionic or nonionic hydrophilic gums, modified cellulosic substances or proteinaceous substances. Examples of such polymers are alkylcelluloses, such as, methylcellulose; hydroxyalkylcelluloses, for example, hydroxymethylcellulose, hydroxyethylcellulose, hydroxypropylcellulose and hydroxybutylcellulose; hydroxyalkyl alkylcelluloses, such as, hydroxyethyl methylcellulose and hydroxypropyl methylcellulose; carboxyalkylcelluloses, such as, carboxymethylcellulose; alkali metal salts of carboxyalkylcelluloses, such as, sodium carboxymethylcellulose; carboxyalkylalkylcelluloses, such as, carboxymethylethylcellulose; carboxyalkylcellulose esters; other natural, semi-synthetic, or synthetic polysaccharides, such as, alginic acid, alkali metal and ammonium salts thereof, carrageenans, galactomannans, tragacanth, agar-agar, gum arabicum, guar gum, xanthan gum, starches, pectins, such as sodium carboxymethylamylopectin, chitin derivates such as chitosan, polyfructans, inulin; polyacrylic acids and the salts thereof; polymethacrylic acids and the salts thereof, methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides such as polyethylene oxide and polypropylene oxide and copolymers of ethylene oxide and propylene oxide.

The hydrophobic polymer suitable for use in the present invention can be selected from the group comprising hydrophobic cellulose derivatives, such as ethyl cellulose, fats, such as glycerol palmitostearate, beeswax, glycowax, castrowax, carnauba wax, glycerol monostearate or stearylalcohol, hydrophobic polyacrylamide derivatives and hydrophobic methacrylic acid derivatives and the like or combinations thereof.

The term “release modifying coating material” means any coating material which modifies the release of the active ingredient in the desired manner. In particular, coating materials suitable for use in the practice of the invention include, but are not limited to, polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit.RTM. RS and RL, poly acrylic acid and poly acrylate and methacrylate copolymers such as those sold under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification) including Eudragit S-100, and Eudragits® NE, RL and RS (water-insoluble polymers having different degrees of permeability and expandability), polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit.RTM. RS-PM, Rohm & Haas), pullulan, collagen, casein, agar, gum arabic, sodium carboxymethyl cellulose, (swellable hydrophilic polymers) poly(hydroxyalkyl methacrylate) (m. wt. .about.5 k-5,000 k), polyvinylpyrrolidone (m. wt. .about.10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. about 30 k-300 k), polysaccharides such as agar, acacia, karaya, tragacanth, alginates and guar, polyacrylamides, Polyox.RTM, polyethylene oxides (m. wt. about 100 k -5,000 k), AquaKeep.RTM. acrylate polymers, diesters of polyglucan, crosslinked polyvinyl alcohol and poly N-vinyl-2-pyrrolidone, sodium starch glucolate (e.g. Explotab.RTM.; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyoxe.RTM., Union Carbide), methyl ethyl cellulose, ethylhydroxy ethylcellulose, cellulose acetate, cellulose butyrate, cellulose propionate, gelatin, collagen, starch, maltodextin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit.RTM., Rohm and Haas), other acrylic acid derivatives, sorbitan esters, natural gums, lecithins, pectin, alginates, ammonium alginate, sodium, calcium, potassium alginates, propylene glycol alginate, agar, and gums such as arabic, karaya, locust bean, tragacanth, carrageenans, guar, xanthan, scleroglucan and mixtures and blends thereof. Combinations of different coating materials may also be used. Multi-layer coatings using different polymers may also be applied. Said release modifying polymeric coating material are preferably contained in a percentage from about 0% to about 20%, more preferably from about 0.2% to about 15% or from about 0.5% to about 10% w/w of the modified release pharmaceutical composition.

Coating composition may optionally comprise further appropriate ingredients which improve the property of coating layers, such ingredients include but are not limited to fillers, plasticizers, anti-adhesive, pigments, coloring agents, stabilizing agents, surfactants, pore formers and the like.

Suitable plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

The following examples are merely illustrative of the present invention and they should not be considered as limiting the scope of the invention in any way, as these examples and other equivalents thereof will become more apparent to those versed in the art in the light of the present disclosure.

### EXAMPLE: 1

### Preparation of Modified Release Capsule composition (Tablets in capsule) of Ramipril 10 mg

Core Composition:

| S. No | Ingredients | Quantity (mg/Tablet) |
|---|---|---|
| 1. | Ramipril | 5.00 |
| 2. | Tromethamine | 10.00 |
| 2. | Calcium sulphate dihydrate | 43.50 |
| 3. | Starch LM 1500 | 15.00 |
| 4. | Sodium Stearyl Fumarate | 1.50 |
| | Total | 75.00 |

Coating Composition:

| S. No. | Ingredients | mg/unit |
|---|---|---|
| 1 | Eudragit S-100 | 05.14 |
| 2 | Talc | 02.57 |
| 3 | Triethyl citrate | 01.28 |
| 4 | Isopropyl alcohol | 56.12 (lost in processing) |
| 5 | Acetone | 56.12 (lost in processing) |
| 6 | Water | 00.02 (lost in processing) |

Process for Preparation:
1. Sift ramipril and other ingredients separately through sieve.
2. Mix ramipril with other ingredients in the geometrical ratio.
3. Compress the blend of step 2 using a tablet compression machine with suitable tooling.
4. Divide the tablets obtained from step 3 into two equal fractions i.e. fraction A & fraction B.
5. Coat the tablets of fraction B with a coating composition.
6. Fill the capsule with one uncoated tablet and one coated tablet

### EXAMPLE: 2

### Preparation of Modified Release Capsule composition (Tablets in capsule) of Ramipril 10 mg

Core Composition:

| S. No | Ingredients | Quantity (mg/Tablet) |
|---|---|---|
| 1. | Ramipril | 05.00 |
| 2. | Calcium sulphate dihydrate | 23.50 |
| 3. | Starch-1500-LM | 35.00 |
| 4. | Magnesium stearate | 01.50 |
| | Total | 65.00 |

Coating composition and the process for preparation of composition is same as that followed in Example: 1

### EXAMPLE: 3

### Preparation of Modified Release Capsule composition (Tablets in capsule) of Ramipril 10 mg

Core Composition:

| S. No | Ingredients | Quantity (mg/Tablet) |
|---|---|---|
| 1. | Ramipril | 05.00 |
| 2. | Ascorbic Acid | 00.50 |
| 2. | Calcium sulphate dihydrate | 53.00 |
| 3. | Starch-1500-LM | 15.00 |
| 4. | Magnesium stearate | 01.50 |
| | Total | 75.00 |

Coating composition and the process for preparation of composition is same as that followed in Example: 1

### EXAMPLE: 4

### Preparation of Modified Release Capsule composition (Tablets in capsule) of Ramipril 10 mg

Core Composition:

| S. No | Ingredients | Quantity (mg/Tablet) |
|---|---|---|
| 1. | Ramipril | 05.00 |
| 2. | Colloidal Silicon Dioxide | 00.50 |
| 2. | Calcium sulphate dihydrate | 53.00 |
| 3. | Starch-1500-LM | 15.00 |
| 4. | Magnesium stearate | 01.50 |
| | Total | 75.00 |

Coating composition and the process for preparation of composition is same as that followed in Example: 1

### EXAMPLE: 5

### Preparation of Modified Release Tablet composition of Ramipril 10 mg

Core Composition:

| | | Quantity |
|---|---|---|
| S. No. | Ingredients | (mg/Tablet) |
| 1. | Ramipril | 10.00 |
| 2. | Calcium sulfate | 75.05 |
| 3. | Hypromellose | 37.50 |
| 4. | Magnesium Stearate | 2.45 |
| Total | | 125.0 |

Process for Preparation:
1. Sift ramipril and other ingredients separately through sieve.
2. Mix ramipril with other ingredients in the geometrical ratio.
3. Compress the blend of step 2 using a tablet compression machine with suitable tooling.
4. Optionally coat the tablet with coating.

In-vitro Test Results:

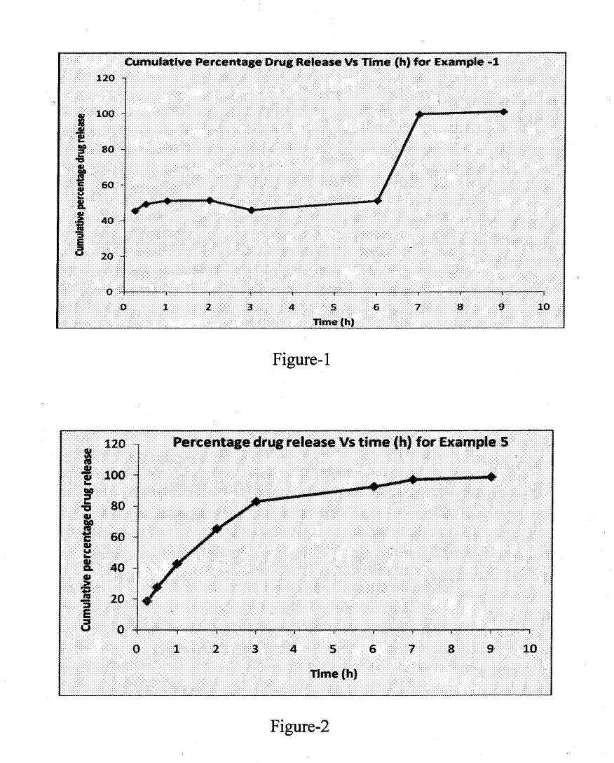

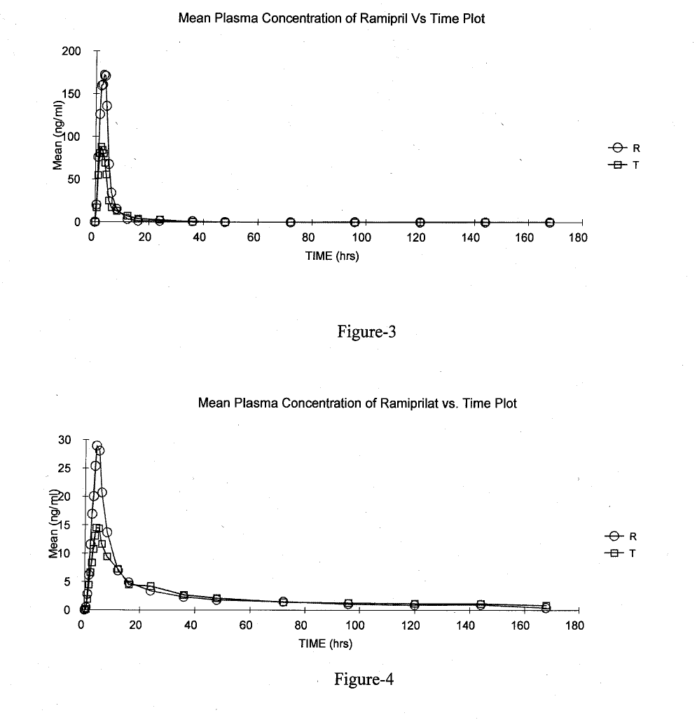

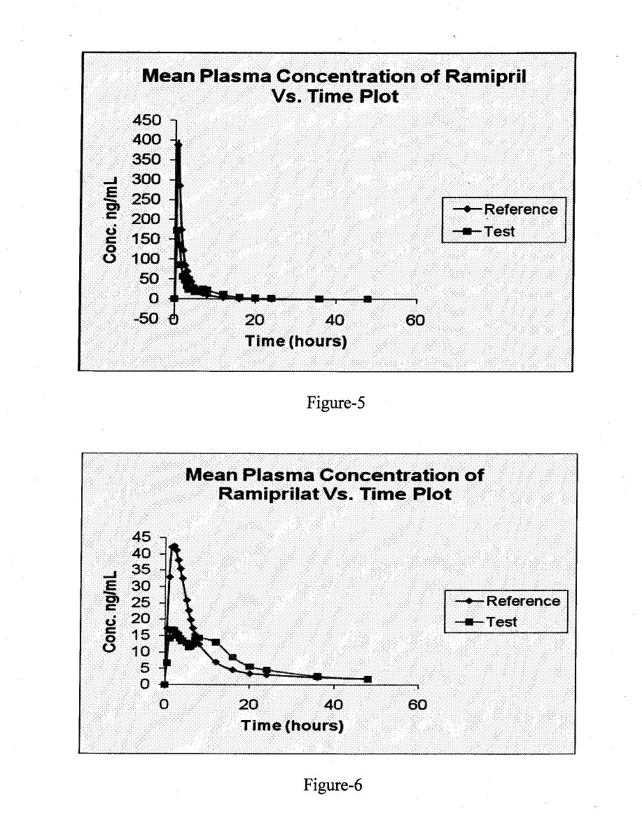

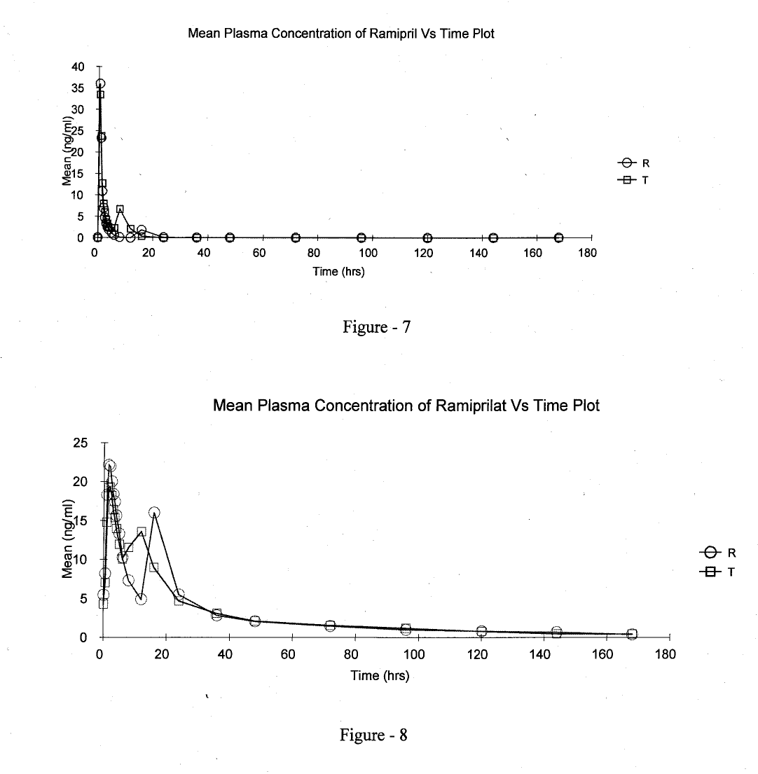

In-vitro dissolution testing were conducted for all the compositions i.e. Example 1 and Example 5 by a pH change dissolution method in USP type II apparatus at 50 rpm. The release profiles of the compositions are shown in FIGS. 1 and 2 respectively.

The drug release data w.r.t time is as follows:

| | | Cumulative % | Cumulative % drug |
|---|---|---|---|
| pH of | Time | drug release | release |
| Medium | (hours) | Example-1 | Example-5 |
| pH 1.2 | 0.25 | 45.42 | 18.50 |
| pH 1.2 | 0.5 | 49.16 | 27.56 |
| pH 1.2 | 1 | 51 | 42.73 |
| pH 1.2 | 2 | 51.41 | 65.27 |
| pH 4.5 | 3 | 45.82 | 82.96 |
| pH 6.8 | 6 | 51.17 | 92.65 |
| pH 7.4 | 7 | 99.73 | 97.16 |
| pH 7.4 | 9 | 101.01 | 98.68 |

Pharmacokinetic Tests:

Three Bio-studies were conducted to compare the rate and extent of absorption of reference product i.e. ALTACE® (Ramipril) Capsules 10 mg of King Pharmaceuticals, USA and the Test product i.e. Ramipril Modified Release Capsules 10 mg manufactured by Panacea Biotec Ltd. (Example-1) , India in healthy adult human subjects.

Single Dose study: Two single dose studies were conducted i.e. one in Fasted condition and one in Fed condition.

Objective of the study: To compare the rate and extent of absorption of single dose of Ramipril Modified Release Capsules 10 mg manufactured by Panacea Biotec Ltd., India (Test Product—Example 1) with single dose of ALTACE® (Ramipril) Capsules 10 mg of King Pharmaceuticals, USA (Reference Product) in healthy, adult, human subjects under fasted and fed conditions and also to measure the average plasma concentration of ramiprilat achieved at 24 hours after single dosing.

Study Design: Open label, balanced, randomized two treatments, two sequences, two period, single dose crossover to compare the pharmacokinetic profiles in 12 healthy, adult, human subjects under fasted and fed conditions.

Dose & Administration: As per the randomization schedule, one capsule of test (T) or reference product (R) will be administered to each subject with 240±2 mL of water at ambient temperature in each period. Subjects will be instructed not to chew or crush the investigational product but to consume it as a whole.

Sampling Schedule:

A total of 22 blood samples will be collected from each subject. The venous blood samples 10 mL each will be withdrawn at pre-dose (before dosing, in the morning of the day of dosing) and 5 mL each at 0.50, 1.00, 1.50, 2.00, 2.50, 3.00, 3.50, 4.00, 5.00, 6.00, 8.00, 12.00, 16.00, 20.00, 24.00, 36.00, 48.00, 72.00, 96.00, 120.00, 144.00 and 168.00 hours after dosing.

Results: Comparative data of the single dose fed and fasted biostudies is as follows:

| | STUDY | | | |
|---|---|---|---|---|
| pKa | FASTED STUDY | | FED STUDY | |
| Parameters | Ramipril | Ramiprilat | Ramipril | Ramiprilat |
| Cmax | 163.24 ± 86.95 | 20.31 ± 7.12 | 102.74 ± 44.87 | 16.01 ± 7.72 |
| (Test) | | | | |
| Tmax₁ | 0.5 | 2.0 | — | — |
| (Test) | | | | |
| in hrs. | | | | |
| Tmax₂ | 7.0 | 8.0 | — | — |
| (Test) | | | | |
| in hrs. | | | | |
| LCmax | 43.72 | 50.40 | 53.34 | 48.87 |
| (Test to | | | | |
| Ref. ratio) | | | | |
| LAUCt | 73.74 | 90.86 | 66.73 | 92.41 |
| (Test to | | | | |
| Ref. ratio) | | | | |
| LAUCinf | 74.50 | 88.91 | 72.61 | 98.96 |
| (Test to | | | | |
| Ref. ratio) | | | | |

Plasma concentration level at 24 hours (ng/mL)

| | | | | |
|---|---|---|---|---|
| Test | 1.047 ± 1.128 | 4.517 ± 1.462 | 4.752 ± 1.266 | 4.233 ± 2.220 |
| Reference | 0.000 ± 0.000 | 3.045 ± 0.915 | 2.290 ± 4.400 | 3.414 ± 0.850 |
| | | | Please clarify | |

It can be seen that the average plasma concentration of ramiprilat at 24 hours after dosing of the Test formulation is higher than the reference product i.e. marketed ramipril formulation (ALTACE®). The plasma concentration of the ramiprilat vs. time is shown in FIGS. 4 and 6 for Fed study and fasting study respectively.

Steady State Steady:

Objective:

Primary: To compare the rate and extent of absorption under steady state level of multiple dose of Ramipril Modified Release Capsules 10 mg manufactured by Panacea Biotec Ltd., India (Test Product i.e. Example-1), with multiple dose of ALTACE® (Ramipril) Capsules 5 mg BID of King Pharmaceuticals, USA (Reference Product) in healthy, adult, human subjects under fasting conditions and also to measure the average plasma concentration of ramiprilat achieved at 24 hours at steady state.

Study Design:

Open label, balanced, randomized, two treatments, two sequences, two period, multiple dose, two-way crossover, comparative pharmacokinetic steady state study in healthy, adult, human subjects under fasting conditions.

Dose and Administration:

As per the randomization schedule, after an overnight fast of at least 9 hrs, either one capsule of test (T) at “0” hour or two capsules of reference product (R) (One capsule of Reference product at “0” hour and one capsule at “12” hours) were administered for 5 days to each subject with 240±2 mL of water at room temperature in each period. Subjects were instructed not to chew or crush the investigational product but to consume it as a whole.

Sampling Schedule:

A total of 43 blood samples were collected from each subject in K₃EDTA vacutainers (containing K₃EDTA as an anticoagulant) during each period.

The venous blood sample 10 mL were withdrawn at pre-dose (before dosing, in the morning of the day of dosing) on Day 1 and 4 mL each were withdrawn at pre-dose (before dosing, in the morning of the day of dosing) for next 4 days. The pre-dose sample of Day 2 and the time point collected at 23.75 hrs post-dose on Day 2 were the same.

The venous blood samples 4 mL each were withdrawn on Day 1 at 0.50, 1.00, 1.50, 2.00, 2.50, 3.00, 3.50, 4.00, 5.00, 5.50, 6.00, 6.50, 7.00, 8.00, 12.00, 16.00, 20.00 and 23.75 hours after dosing and on Day 5 onwards at 0.50, 1.00, 1.50, 2.00, 2.50, 3.00, 3.50, 4.00, 5.00, 6.00, 8.00, 12.00, 16.00, 24.00, 36.00, 48.00, 72.00, 96.00, 120.00, 144.00 and 168.00 hours after dosing. Note: The 12.00 hour blood sample on Day 1 and on Day 5 was collected within 2 mins of the scheduled sampling time to ensure dosing of the second dose of the reference product.

Pharmacokinetic Results for Ramiprilat

| Parameters (Units) | Test (T) (Mean ± SD) | Reference (R) (Mean ± SD) |
|---|---|---|
| Cₘₐₓ (ng/mL) | 21.2487 ± 5.41728 | 23.3654 ± 5.77890 |
| AUCₜₐᵤ (ng · hr/ml) | 255.2129 ± 62.46277 | 264.6880 ± 53.22971 |
| Tₘₐₓ (hr) | 4.8131 ± 4.44363 | 2.6923 ± 4.00800 |
| Cmin(1/hr) | 4.0789 ± 1.46458 | 4.7615 ± 1.21489 |
| Plasma | 4.67 ± 0.40 | 5.49 ± 0.42 |
| concentration of | | |
| Ramiprilat at 24 | | |
| hours (ng/mL) | | |
| ACE Activity | Below LLOQ in | Below LLOQ in |
| | 61.53% subjects | 69.23% subjects |

Summary statistics of Log transformed Pharmacokinetic
parameters for Ramiprilat

| Product/Statistics | Cmax | AUCₜₐᵤ | Cmin |
|---|---|---|---|
| Ratio of least square | 92.4 | 95.9 | 84.4 |
| mean T/R (%) | | | |

The plasma concentration of the ramiprilat vs. time is shown in FIG. 8 for steady state study.

1. A modified release pharmaceutical composition comprising ramipril or pharmaceutically acceptable salts thereof and pharmaceutically acceptable excipient(s) wherein pharmaceutical composition is bioequivalent to conventional immediate release formulation of ramipril administered twice daily.

2. A modified release pharmaceutical composition according to claim 1 wherein the composition provides extended therapeutic plasma levels.

3. A modified release pharmaceutical composition according to claim 1 wherein the composition provides a peak plasma concentration of ramiprilat of at least about 4 ng/ml to about 40 ng/ml after administration of 10 mg ramipril.

4. A modified release pharmaceutical composition according to claim 1 wherein the composition exhibits the following dissolution profile: not more than 80% of drug is released in 2 hours and at least about 15% of drug is released within 6 hours, when tested by a pH Change dissolution method in USP Type II apparatus at 50 rpm.

5. A modified release pharmaceutical composition according to claim 1 wherein the composition exhibits the following dissolution profile: not more than 65% of drug is released in 2 hours and at least about 30% of drug is released within 6 hours, when tested by a pH Change dissolution method in USP Type II apparatus at 50 rpm.

6. A modified release pharmaceutical composition according to claim 1 wherein the composition exhibits the following dissolution profile: at least about 40% of drug is released in 3 hours and not more than 60% of drug is released in 3 hours, when tested by a pH Change dissolution method in USP Type II apparatus at 50 rpm.

7. A modified release pharmaceutical composition according to claim 1 wherein the composition exhibits the following dissolution profile: at least about 40% of drug is released within 2 hours and not more than 60% of drug is released in 3 hours, when tested by a pH Change dissolution method in USP Type II apparatus at 50 rpm.

8. A modified release pharmaceutical composition according to claim 1 wherein the composition exhibits the following dissolution profile: at least about 30% of drug is released within 30 min, not more than 55% of drug is released in 5 hours and atleast about 80% is released at 9 hours, when tested by a pH Change dissolution method in USP Type II apparatus at 50 rpm.

9. A modified release pharmaceutical composition according to claim 1 wherein the composition is selected from the group consisting of sustained release, delayed release, pulsed release, controlled release, extended release, immediate release and combinations thereof.

10. A modified release pharmaceutical composition according to claim 9 wherein the composition is sustained release composition or a pulsed release composition.

11. A modified release pharmaceutical composition according to claim 1 wherein the composition comprises ramipril or pharmaceutically acceptable salts thereof in an amount from about 1% to 40% w/w of the composition and one or more pharmaceutically acceptable excipient in an amount from about 1% to about 99% w/w of the composition selected from the group consisting of diluents, carriers, fillers, release modifying material, bulking agents, binders, disintegrants, polymer, lubricant, glidant, surface active agents, stabilizers, absorption accelerators, flavoring agents, preservatives, antioxidants, and buffering agents.

12. A modified release pharmaceutical composition comprising:
a) an immediate release component comprising ramipril or pharmaceutically acceptable salts thereof and one or more pharmaceutically acceptable excipient;
b) a modified release component comprising ramipril or pharmaceutically acceptable salts thereof and one or more pharmaceutically acceptable excipient; and
optionally a sustained release component comprising ramipril or pharmaceutically acceptable salt thereof and one or more pharmaceutically acceptable excipient.

13. A modified release pharmaceutical composition according to claim 12 wherein the immediate release component comprises ramipril or pharmaceutically acceptable salts thereof in an amount from about 30 to about 80% w/w of the total amount of ramipril in the modified release composition and modified release component comprises ramipril or pharmaceutically acceptable salts thereof in an amount from about 20% to about 70% w/w of the of the total amount of ramipril in the modified release composition.

14. A modified release pharmaceutical composition according to claims 1 wherein the pharmaceutically acceptable excipient is selected from the group consisting of diluents, carriers, fillers, release modifying material, bulking agents, binders, disintegrants, polymer, lubricant, glidant, surface active agents, stabilizers, absorption accelerators, flavoring agents, preservatives, antioxidants, buffering agents and combinations thereof.

15. A modified release pharmaceutical composition according to claim 14 wherein the diluent is selected from the group consisting of calcium carbonate, calcium phosphate (dibasic or tribasic), calcium sulfate anhydrous, calcium sulfate hydrate, dextrates, dextrin, dextrose, fructose, kaolin, lactitol, anhydrous lactose, lactose monohydrate, maltose, mannitol, sorbitol, sucrose, starch, pregelatinized starch and combinations thereof.

16. A modified release pharmaceutical composition according to claim 15 wherein the diluent is calcium sulfate dihydrate.

17. A modified release pharmaceutical composition according to claim 14 wherein the disintegrant is selected from the group consisting of starch, pregelatinized starch, sodium starch glycolate, sodium carboxymethyl cellulose, crosslinked sodium carboxymethyl cellulose, clays, microcrystalline cellulose, alginates, gums, surfactants, effervescent mixtures, hydrous aluminum silicate, cross-linked polyvinyl pyrrolidone and combinations thereof.

18. A modified release pharmaceutical composition according to claim 17 wherein the disintegrant is pregelatinized starch.

19. A modified release pharmaceutical composition according to claim 14 wherein the lubricant is selected from the group consisting of calcium stearate, glyceryl behenate, magnesium stearate, mineral oil, polyethylene glycol, sodium stearyl fumarate, stearic acid, talc, vegetable oil, sodium lauryl sulfate, zinc stearate and combinations thereof.

20. A modified release pharmaceutical composition according to claim 19 wherein the lubricant is magnesium stearate.

21. A modified release pharmaceutical composition according to claim 14 wherein the stabilizer is selected from the group consisting of buffering agents including tromethamine, alkalizing agents, amino acid or its salts, cellulose or its derivatives, colloidal silicon dioxide, silica, ascorbic acid, organic acids, glutamic acid, betaine hydrochloride, tartaric acid and combinations thereof.

22. A modified release pharmaceutical composition according to claim 21 wherein the stabilizer is a buffering agent.

23. A modified release pharmaceutical composition according claim 1 wherein the ramipril is present in an amount from about 2.5 mg to about 20 mg.

24. A modified release pharmaceutical composition according claim 1 wherein the composition is formulated into a dosage form selected from the group consisting of tablets, capsules, mini-tablets, oral films, sprays, pellets, granules, powers, and combinations thereof.

25. A modified release pharmaceutical composition according claim 1 wherein the composition provides the average plasma concentration of ramipril at of at least about 4 ng/ml at 24 hours after single dose administration of 10 mg ramipril.

26. A modified release pharmaceutical composition according to claim 12 wherein the composition exhibits Tₘₐₓ₁ ranging from about 1 hour to about 4 hours and Tₘₐₓ₂ ranging from about 6 hours to about 10 hours.

27. A modified release pharmaceutical composition comprising ramipril or pharmaceutically acceptable salts thereof and a release modifying material and optionally one or more pharmaceutically acceptable excipient(s), wherein the composition provides average plasma concentration of ramiprilat at 24 hours comparable with that of the marketed ramipril formulation administered twice daily.

28. A modified release pharmaceutical composition of claim 27 wherein the composition provides the average plasma concentration of ramiprilat of atleast about 4.0 ng/ml at 24 hours after administration of 10 mg ramipril at steady state.
